(19) United States
(12) Patent Application Publication
(10) Pub. No.: US 2009/0088442 A1
(43) Pub. Date: Apr. 2, 2009

(54) PROPHYLACTIC/THERAPEUTIC AGENT FOR ABNORMALITIES OF SUGAR/LIPID METABOLISM

(75) Inventors: Yuji Abe, Osaka (JP); Aki Kusunoki, Osaka (JP); Yoshiharu Hayashi, Osaka (JP); Fumihiko Akahoshi, Osaka (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MITSUBISHI TANABE PHARMA CORPORATION, Osaka-shi, Osaka (JP)

(21) Appl. No.: 11/912,717

(22) PCT Filed: Apr. 26, 2006
(86) PCT No.: PCT/JP2006/308695

§ 371 Date: Nov. 26, 2007

(30) Foreign Application Priority Data
Apr. 26, 2005 (JP) 2005-127523

(51) Int. Cl. A61K 31/496; C07D 413/14; A61P 3/08; A61P 3/06

(52) U.S. Cl. 514/254.02; 544/369

(57) ABSTRACT

The present invention provides a pharmaceutical agent for the treatment and/or prophylaxis of abnormal blood glucose and lipid metabolism associated with eating, for which a sufficient treatment method or a therapeutic drug has not been found.

### TECHNICAL FIELD

The present invention relates to a pharmaceutical agent for the treatment and/or prophylaxis of abnormal blood glucose and lipid metabolism.

### BACKGROUND ART

Hyperlipidemia refers to a condition where cholesterol or neutral fat in the blood has abnormally increased, which is one of the important risk factors of the onset of arteriosclerotic diseases such as ischemic heart disease and the like.

In recent years, it has been reported that, in addition to the increased blood lipid level, diabetes and hyperinsulinemia are important risk factors of the onset of arteriosclerotic diseases (see, non-patent reference 1). Particularly, when plural risk factors such as abnormal lipid metabolism, abnormal glucose metabolism, obesity, hypertension and the like are observed, the risk of arteriosclerotic disease of the subject is considered to increase strikingly. The pathology associated with plural risk factors is attracting attention as “metabolic syndrome” or “multiple risk factor syndrome”.

As the diagnostic criteria of metabolic syndromes, several healthcare organizations have proposed diagnostic criteria in recent years, such as those of World Health Organization (WHO), US treatment guideline for hyperlipidemia and the like.

For example, according to the diagnostic criteria of WHO, when a subject shows at least one of type 2 diabetes, impaired glucose tolerance and insulin resistance, and falls under at least two of an increased blood pressure (≧160/90 mmHg), increased plasma neutral fat (not less than 150 mg/dL and/or HDL cholesterol low value of less than 35 mg/dL for male, less than 39 mg/dL for female), central obesity (the ratio of waist to hip exceeding 0.90 for male, exceeding 0.85 for female and/or BMI exceeding 30 kg/m²), and a trace amount of albumin urine (urinary albumin excretion rate of not less than 20 μg/min, or the ratio of albumin:creatinine of not less than 30 mg/g), the subject is diagnosed to have a metabolic syndrome (see, non-patent reference 2).

According to US treatment guidelines for hyperlipidemia (NCE-ATPIII: National Cholesterol Education Program Adult Treatment Panel III), when a subject falls under at least three of visceral fat type obesity (waist size exceeding 102 cm for male, 88 cm for female), increased neutral fat (blood triglyceride (hereinafter to be referred to as TG) concentration of not less than 150 mg/dL), decrease in HDL cholesterol (less than 40 mg/dL for male, less than 50 mg/dL for female), blood pressure increase (systolic blood pressure is not less than 130 mmHg, or diastolic blood pressure is not less than 85 mmHg), and blood glucose increase (fasting blood sugar level is not less than 110 mg/dL), the subject is diagnosed with a metabolic syndrome (see, non-patent reference 3).

While diagnostic criteria of metabolic syndrome partly differ between WHO and US guideline for hyperlipidemia, they are common in that obesity, hypertension, borderline diabetes, hypertriglyceridemia and low high-density lipoprotein cholesterol are important risk factors. Therefore, for the prophylaxis or treatment of arteriosclerotic diseases, it is important to control LDL cholesterol to an adequate level as well as comprehensively manage risk factors because, in metabolic syndrome, for example, abnormal lipid metabolism and abnormal glucose metabolism are observed in combination.

The management goal of blood lipid level varies depending on the presence or absence of previous ischemic heart disease and the presence or absence of risk factors (complications of hypertension, diabetes etc.) other than lipid. When most strict treatment is required, the total cholesterol is not more than 200 mg/dL, LDL cholesterol is not more than 100 mg/dL, HDL cholesterol is not less than 40 mg/dL, and TG is not more than 150 mg/dL (see, non-patent reference 3).

The main characteristics of pathologically abnormal glucose and lipid metabolism represented by a metabolic syndrome and the like are an increase in neutral fat and blood glucose levels after eating, which are called postprandial hyperlipidemia and postprandial hyperglycemia, respectively. The main blood lipid that increases after eating is TG. Consequently, VLDL rich in TG increases in blood, decreases HDL and increases the risk of arteriosclerosis (see, non-patent reference 4). Moreover, postprandial hyperlipidemia and postprandial hyperglycemia independently and additively cause oxidative stress in the vascular endothelium, increasing the risk of arteriosclerosis (see, non-patent reference 5).

While a lipid-lowering drug or a blood glucose-lowering drug is used for the treatment of abnormal glucose and lipid metabolism, the effects of these pharmaceutical agents are not entirely satisfactory. For example, while HMG-CoA reductase inhibitor affords a superior LDL cholesterol-lowering effect, it offers little hope for a blood glucose level-improving effect. In addition, while insulin sensitizer affords a good influence on blood glucose and TG, it adversely influences cardiac failure because it causes body weight gain and edema. In consideration of the above, careful medication management is demanded (see, non-patent reference 6).

A report has documented that a rapid-acting insulin secretagogue, nateglinide [(−)-N-(trans-4-isopropylcyclohexylcarbonyl)-D-phenylalanine], suppresses increase in blood lipid by fat loading in rats with type 2 diabetes (see, non-patent reference 7). However, since insulin secretagogue such as nateglinide possibly causes hypoglycemia, it requires strict medication management in line with the mealtime. Therefore, the drug is not necessary a satisfactory treatment method of postprandial hyperlipidemia and postprandial hyperglycemia.

Glucagon-like peptide-1 (hereinafter to be referred to as GLP-1) and glucose-dependent insulinotropic polypeptide (hereinafter to be referred to as GIP), which are secreted from the gastrointestinal tract after eating, have a strong insulin secretagogue action. However, since GLP-1 and GIP are degraded by dipeptidyl peptidase IV (hereinafter to be referred to as DPP-IV), they may not be able to sufficiently act in the body. DPP-IV inhibitor promotes secretion of insulin by suppressing degradation of GLP-1 and GIP and shows a hypoglycemic action. Therefore, the development thereof as a therapeutic drug for type 2 diabetes is ongoing (see, non-patent reference 8). Nevertheless, an abnormal lipid metabolism-improvement effect based on DPP-IV inhibitory action is not developed actively.

non-patent reference 1: Isomaa B, Almgren P, Tuomi T, Forsen B, Lahti K, Nissen M, Taskinen M. R., Groop L: Diabetes Care 2001; 24: 683-689.
non-patent reference 2: Alberti K. G., Zimmet P. Z.: Diabet Med 1998; 15: 539.
non-patent reference 3: National Institutes of Health: Third Report of the National Cholesterol Education Program Expert Panel on Detection, Evaluation, and Treatment of High Blood Cholesterol in Adults (Adult Treatment Panel III). Executive Summary. Bethesda, Md., National Institutes of Health, National Heart, Lung and Blood Institute, 2001 (NIH publ. no. 01-3670)
non-patent reference 4: Carr, M. C., Brunzell, J. D.: J Clin Endoclinol Metab Circ 2004; 89: 2601-2607.
non-patent reference 5: Ceriello A., Taboga C., Tonutti L., Quagliaro L., Piconi L., Bais B., Ros R. D., Motz E.: Circulation 2002; 106: 1211-1218.
non-patent reference 6: Nesto R. W., Bell D., Bonow R. O., Fonseca V., Grundy S. M., Horton E. S., Winter M. L., Porte D., Semenkovich C. F., Smith S., Young L. H., Kahn R.: Circulation 2003; 108: 2941-2948.
non-patent reference 7: Mine T., Miura K., Kitahara Y., Okano A., Kawamori R.: Biol Pharm Bull. 2002; 25: 1412-1416.
non-patent reference 8: Weber A. E.: J. Med. Chem. 2004; 47: 4135-4141.

### DISCLOSURE OF THE INVENTION

### Problems to be Solved by the Invention

The problem of the present invention is to provide a pharmaceutical agent for the prophylaxis and/or treatment of abnormal glucose and lipid metabolism, for which a sufficient treatment method and a therapeutic drug have not been found, particularly, abnormal glucose and lipid metabolism associated with eating.

### Means of Solving the Problems

The present inventors have studied in view of the above-mentioned problems and found that 3-{(2S,4S)-4-[4-(3-methyl-1-phenyl-1H-pyrazol-5-yl)piperazin-1-yl]pyrrolidin-2-ylcarbonyl}thiazolidine hydrobromide (hereinafter to be also referred to as compound 1) suppresses an increase in the plasma TG concentration after fat loading in obese Zucker fatty rat having insulin resistance (hereinafter to be referred to as ZF rat). They have studied the above result in more depth and found that, in addition to TG, the blood glucose level also increases when fat is loaded, and that compound 1 suppresses blood glucose increase after the fat loading, and further, markedly suppresses blood glucose increase after glucose loading in an oral glucose loading test after fat loading. Moreover, they have found that, unlike insulin secretagogue, compound 1 does not induce hypoglycemia, which resulted in the completion of the present invention.

### EFFECT OF THE INVENTION

The pharmaceutical agent of the present invention can simultaneously suppress a postprandial increase of blood TG and glucose observed in metabolic syndrome and the like with a single pharmaceutical agent. Unlike insulin secretagogue (e.g., nateglinide) and the like, the pharmaceutical agent of the present invention can be used safely without causing hypoglycemia. Moreover, it can be easily used in combination with other agents, and can correct abnormal lipid metabolism and abnormal glucose metabolism by a combined use of the pharmaceutical agent of the present invention and a general lipid-lowering drug, even when a decrease in lipid and blood glucose cannot be expected with a general lipid-lowering drug alone.

That is, the compound of the present invention is effective as a pharmaceutical agent for the prophylaxis and/or treatment of abnormal glucose and lipid metabolism associated with diet, that is, postprandial hyperglycemia and postprandial hyperlipidemia and the like.

### BRIEF DESCRIPTION OF THE DRAWINGS

FIG. 1 shows the suppressive action of compound 3 on an increase in plasma TG after oral fat loading in ZF rat, where the plot at each time point shows mean±standard error. *P<0.05, **P<0.01: comparison with vehicle group (Student's t-test)

FIG. 2 shows the suppressive action of compound 3 on an increase in plasma free fatty acids after oral fat loading in ZF rat, where the plot at each time point shows average value±standard error. *P<0.05, **P<0.01: comparison with vehicle group (Student's t-test)

FIG. 3 shows the suppressive action of compound 3 on an increase in plasma glucose after oral fat loading in ZF rat, where the plot at each time point shows mean±standard error. *P<0.05, **P<0.01: comparison with vehicle group (Student's t-test)

FIG. 4 shows the action of compound 3 on the concentration of plasma insulin after oral fat loading in ZF rat, where the plot at each time point shows mean±standard error. **P<0.01: comparison with vehicle group (Student's t-test)

FIG. 5 shows the suppressive action of compound 3 on an increase in the concentration of plasma glucose after oral glucose loading in fat-loaded ZF rat, where the plot at each time point shows average value±standard error. **P<0.01: comparison with vehicle group (Student's t-test)

FIG. 6 shows the influence of compound 3 and nateglinide on the concentration of plasma glucose in overnight-fasted Wistar rat, where each column shows mean±standard error. **P<0.01: comparison with vehicle group (Dunnett's multiple comparison test)

FIG. 7 shows the influence of compound 3 and nateglinide on the concentration of plasma insulin in Wistar rat fasted overnight, where each column shows mean±standard error. **P<0.01: comparison with vehicle group (Dunnett's multiple comparison test)

### BEST MODE FOR EMBODYING THE INVENTION

That is, the present invention relates to pharmaceutical agents for the prophylaxis and/or treatment of the following (1) to (6).

(1) A pharmaceutical agent for the prophylaxis and/or treatment of abnormal glucose and lipid metabolism, which comprises, as an active ingredient, a salt of 3-{(2S,4S)-4-[4-(3-methyl-1-phenyl-1H-pyrazol-5-yl)piperazin-1-yl]pyrrolidin-2-ylcarbonyl}thiazolidine with an organic or inorganic mono- or di-basic acid, or a solvate thereof.
(2) The pharmaceutical agent for the prophylaxis and/or treatment of the above-mentioned (1), wherein the abnormal glucose and lipid metabolism is metabolic syndrome, hyperlipidemia, diabetic hyperlipidemia, postprandial hyperlipidemia or postprandial hyperglycemia.
(3) The pharmaceutical agent for the prophylaxis and/or treatment of the above-mentioned (1), wherein the organic or inorganic monobasic acid is hydrochloric acid, hydrobromic acid, nitric acid, mesyl acid, tosyl acid, besyl acid, hydrochloric acid, naphthalene-1-sulfonic acid, naphthalene-2-sulfonic acid, gallic acid or camphorsulfonic acid.
(4) The pharmaceutical agent for the prophylaxis and/or treatment of the above-mentioned (1), wherein the organic or inorganic mono- or di-basic acid is 2.0 hydrobromic acid, 2.5 hydrobromic acid, 2 maleic acid, 2 tosyl acid, 2.5 hydrochloric acid, 2 naphthalene-1-sulfonic acid, 2 mesyl acid, 3 mesyl acid or 2 naphthalene-2-sulfonic acid.
(5) A pharmaceutical agent for the prophylaxis and/or treatment of abnormal glucose and lipid metabolism, which comprises, as an active ingredient, 3-{(2S,4S)-4-[4-(3-methyl-1-phenyl-1H-pyrazol-5-yl)piperazin-1-yl]pyrrolidin-2-ylcarbonyl}thiazolidine 2.5 hydrobromide or a solvate thereof.
(6) The pharmaceutical agent for the prophylaxis and/or treatment of the above-mentioned (5), wherein the abnormal glucose and lipid metabolism is metabolic syndrome, hyperlipidemia, diabetic hyperlipidemia, postprandial hyperlipidemia or postprandial hyperglycemia.

While the definitions of the terms used in the present specification are described in the following, the following definitions do not limit the scope of the present invention.

The “mono- or di-basic acid” is an acid capable of affording one or two protons, and the mono- or di-basic acid may be an organic acid or an inorganic acid. As the “organic or inorganic mono- or di-basic acid”, hydrochloric acid, hydrobromic acid, nitric acid, mesyl acid, tosyl acid, besyl acid, hydrochloric acid, naphthalene-1-sulfonic acid, naphthalene-2-sulfonic acid, gallic acid or camphorsulfonic acid and the like can be mentioned, and hydrobromic acid, maleic acid, tosyl acid, hydrochloric acid, naphthalene-1-sulfonic acid, mesyl acid, mesyl acid, or 2 naphthalene-2-sulfonic acid are preferable.

The “solvate” is a compound wherein a solvent is bonded. When the solvent is water, it may be particularly indicated as a hydrate. The salt as an active ingredient in the pharmaceutical agent of the present invention may be present as any solvate, and a hydrate is more preferable.

The “abnormal glucose and lipid metabolism” means a condition where some abnormality occurs in the carbohydrate or lipid metabolism pathway (including absorption), and the blood concentration is not maintained in an appropriate range (mostly beyond the normal blood concentration range). It is a pathological state requiring a treatment according to the diagnostic criteria such as US hyperlipidemia guideline, WHO guideline and the like. Specifically, metabolic syndrome, hyperlipidemia, diabetic hyperlipidemia, postprandial hyperlipidemia, or postprandial hyperglycemia and the like can be mentioned.

The “3-{(2S,4S)-4-[4-(3-methyl-1-phenyl-1H-pyrazol-5-yl)piperazin-1-yl]pyrrolidin-2-ylcarbonyl}thiazolidine (hereinafter to be referred to as compound 2)” is a compound represented by the following chemical formula (2).

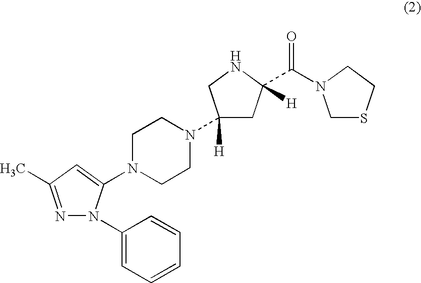

The 3 hydrochloride of compound 2 can be produced according to the synthesis method described as Example 222 of WO02/14271. In addition, this can be converted to compound 2 using a suitable base.

A salt of compound 2 with an organic or inorganic mono- or di-basic acid, and a solvate thereof, which are active ingredients of the pharmaceutical agent of the present invention, are various novel salt forms of compound 2 described in the above-mentioned patent description, which are afforded according to a conventional method.

The pharmaceutical agent of the present invention can be administered orally or parenterally (intravenously, subcutaneously etc.) in a general administration form (tablet, capsule, powder etc.). The pharmaceutical agent of the present invention is desirably administered once a day or several times a day in consideration of in vivo stability and bioavailability. Such dose range is 0.01 mg-100 mg per 1 kg of body weight.

The present invention is explained in detail in the following by referring to Experimental Examples, which are not to be construed as limitative. The “compound 3” used in the following Experimental Examples is a hydrate of 2.5 hydrobromide of the aforementioned compound 2.

### EXAMPLES

### Experimental Example 1

### Effect of Compound 3 on Abnormal Lipid Metabolism and Abnormal Glucose Metabolism After Oral Fat Loading in ZF Rat

### (Test Method)

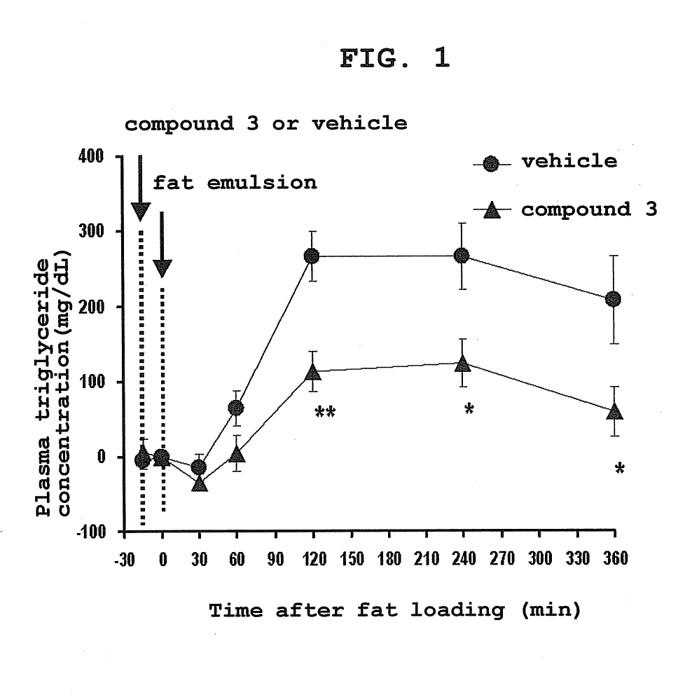

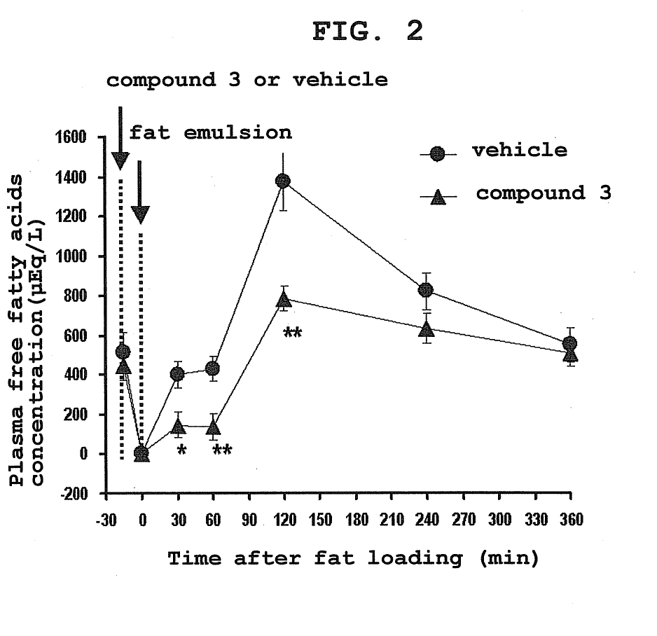

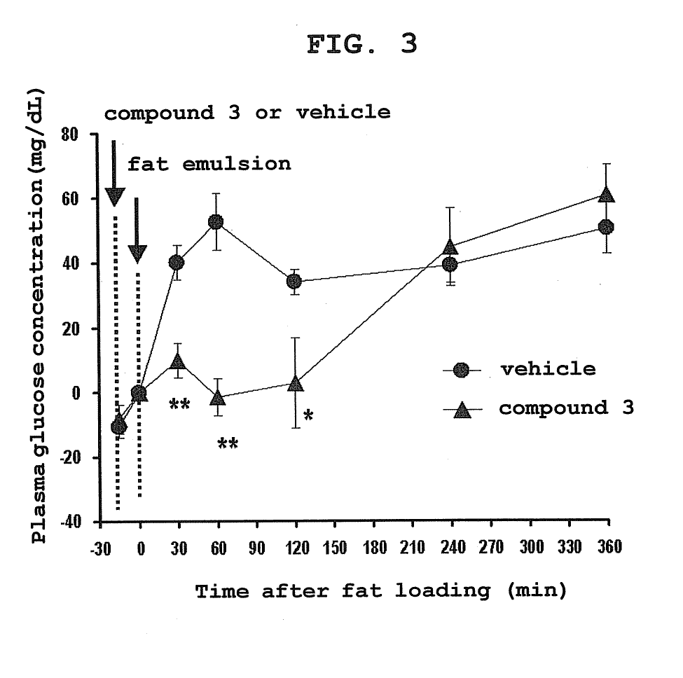

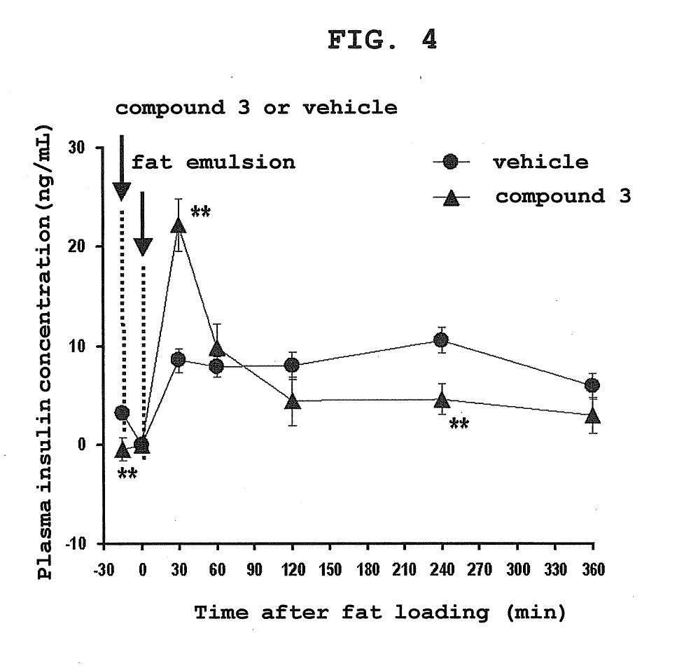

The test was performed using male ZF rats. The rats were divided into two groups (10 rats/group). Compound 3 (1 mg/kg) or a 0.5% hydroxypropylmethylcellulose solution, which was a vehicle used to dissolve the compound, was administered by gavage to each of the rats. The administered volume was 2 mL/kg for both. At 15 min after the administration, a fat emulsion (main component was soybean oil, Intralipos; Otsuka Pharmaceutical Factory, Inc.) was orally loaded at the rate of 10 mL/kg. Blood samples were sequentially collected, and plasma TG concentration, free fatty acids concentration, glucose concentration and insulin concentration were measured. The amount of change from the value before fat loading in each index is shown in FIG. 1 to FIG. 4.

### (Results)

In ZF rat, the plasma TG concentration continuously increased until 6 hr after fat loading. Compound 3 suppressed an increase in the plasma TG concentration and free fatty acids concentration after fat loading. In addition, an increase in the concentration of plasma glucose by fat loading was observed in ZF rat. Compound 3 also suppressed the increase in the plasma glucose concentration after fat loading. Moreover, compound 3 transiently increased insulin concentration after fat loading.

### Experimental Example 2

### Effect of Compound 3 on Increase in Plasma Glucose after Oral Glucose Loading in Fat-Loaded ZF Rat

### (Test Method)

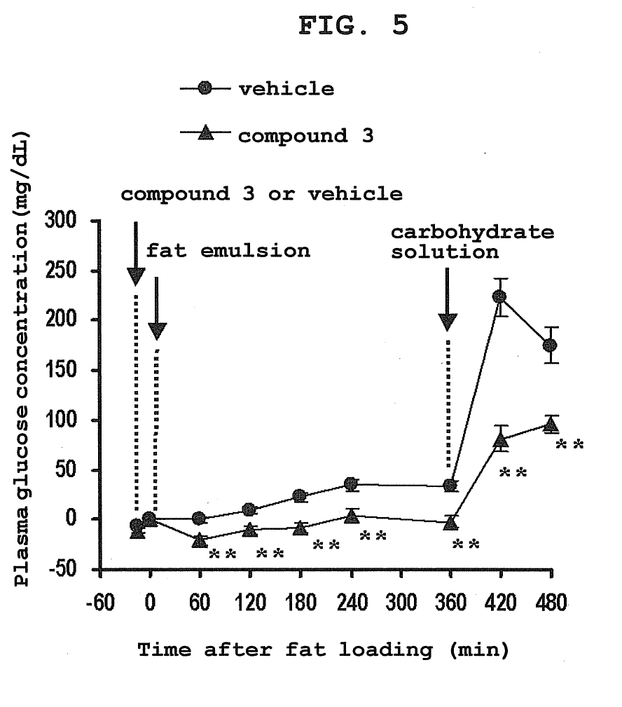

The test was performed using male ZF rats. The number of experimental examples was 10 for each group. Compound 3 (1 mg/kg) or a vehicle was administered by gavage to each of the rats. The administered volume was 2 mL/kg for both. At 15 min after the administration, a fat emulsion (main component was soybean oil, Intralipos; Otsuka Pharmaceutical Factory, Inc.) was orally loaded at the rate of 2 mL/kg. Furthermore, at 6 hr after the administration of fat emulsion, a mixed carbohydrate solution of starch, sucrose and lactose (mixing ratio 6:3:1) was orally administered at 3.5 g/kg. The volume administered of each of the fat emulsion and the carbohydrate solution was 10 mL/kg. Blood samples were sequentially collected, and plasma glucose concentration was measured. The amount of change in plasma glucose from the value before fat loading is shown in FIG. 5.

### (Results)

Compound 3 suppressed an increase in the baseline plasma glucose concentration by fat loading in fat-loaded ZF rat, and suppressed an increase in the plasma glucose concentration after oral glucose loading in an oral glucose loading test performed at 6 hr after fat loading.

### Experimental Example 3

### Effect of Compound 3 on Fasting Blood Sugar Level in Wistar Rat

### (Test Method)

The test was performed using male Wistar rats. The rats were fasted over night and, after fasting, divided into groups (8 rats/group). A compound 3 solution, a nateglinide suspension or a vehicle was administered by gavage to each of them. The dose of compound 3 was 0.01, 0.1, 1, 10 or 100 mg/kg. The dose of nateglinide was 10, 30, 100 or 300 mg/kg. The volume administered was 2 mL/kg in all cases.

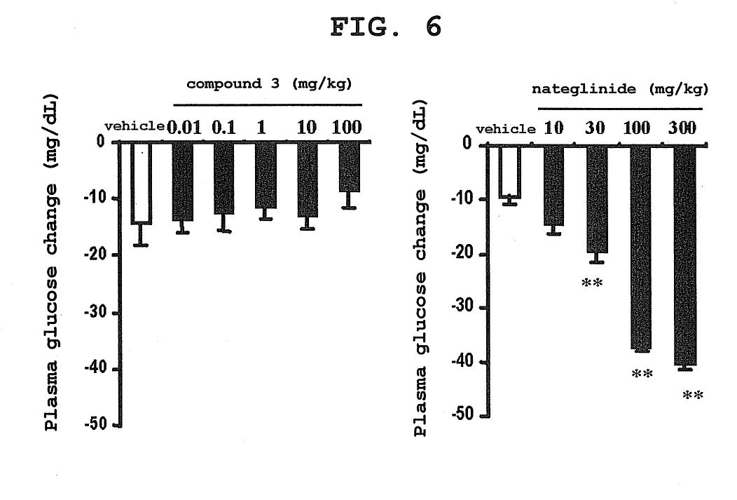

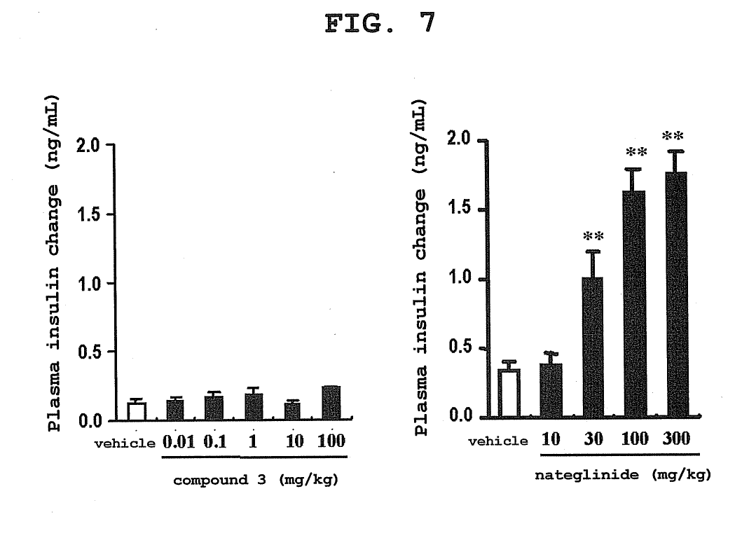

Blood samples were sequentially collected, and plasma glucose concentration and insulin concentration were measured. The maximum amount of change in each index from the value before drug administration is shown in FIG. 6 and FIG. 7.

### (Results)

Compound 3 showed no effect on fasting plasma glucose concentration and fasting insulin concentration in Wistar rats at the dose of 100 mg/kg. In contrast, nateglinide increased plasma insulin concentration and induced hypoglycemia.

### INDUSTRIAL APPLICABILITY

A pharmaceutically acceptable salt and the like of 3-{(2S,4S)-4-[4-(3-methyl-1-phenyl-1H-pyrazol-5-yl)piperazin-1-yl]pyrrolidin-2-ylcarbonyl}thiazolidine are effective as agents for the treatment and/or prophylaxis of abnormal blood glucose and lipid metabolism associated with eating and promote the development of pharmaceutical products.

This application is based on a patent application No. 2005-127523 filed in Japan, the contents of which are incorporated in full herein by this reference.

1. A pharmaceutical agent for the prophylaxis and/or treatment of abnormal glucose and lipid metabolism, which comprises, as an active ingredient, a salt of 3-{(2S,4S)-4-[4-(3-methyl-1-phenyl-1H-pyrazol-5-yl)piperazin-1-yl]pyrrolidin-2-ylcarbonyl}thiazolidine with an organic or inorganic mono- or di-basic acid, or a solvate thereof.

2. The pharmaceutical agent of claim 1, wherein the abnormal glucose and lipid metabolism is metabolic syndrome, hyperlipidemia, diabetic hyperlipidemia, postprandial hyperlipidemia or postprandial hyperglycemia.

3. The pharmaceutical agent of claim 1, wherein the organic or inorganic monobasic acid is hydrochloric acid, hydrobromic acid, nitric acid, mesyl acid, tosyl acid, besyl acid, naphthalene-1-sulfonic acid, naphthalene-2-sulfonic acid, gallic acid or camphorsulfonic acid.

4. The pharmaceutical agent of claim 1, wherein the organic or inorganic mono- or di-basic acid is 2.0 hydrobromic acid, 2.5 hydrobromic acid, 2 maleic acid, 2 tosyl acid, 2.5 hydrochloric acid, 2 naphthalene-1-sulfonic acid, 2 mesyl acid, 3 mesyl acid or 2 naphthalene-2-sulfonic acid.

5. A pharmaceutical agent for the prophylaxis and/or treatment of abnormal glucose and lipid metabolism, which comprises, as an active ingredient, 3-{(2S,4S)-4-[4-(3-methyl-1-phenyl-1H-pyrazol-5-yl)piperazin-1-yl]pyrrolidin-2-ylcarbonyl}thiazolidine 2.5 hydrobromide, or a solvate thereof.

6. The pharmaceutical agent of claim 5, wherein the abnormal glucose and lipid metabolism is metabolic syndrome, hyperlipidemia, diabetic hyperlipidemia, postprandial hyperlipidemia or postprandial hyperglycemia.
